(19) United States
(12) Patent Application Publication
(10) Pub. No.: US 2004/0071650 A1
(43) Pub. Date: Apr. 15, 2004

(54) Use of cyclosporin 7-thioamide derivatives for hair growth

(76) Inventors: Sang-Nyun Kim, Yusong-gu (KR); Ho-Jeong Ahn, Yusong-gu (KR); Chang-Woo Lee, Seo-gu (KR); Seung-Jin Kim, Seoul (KR); Min-Ho Lee, Yusong-su (KR); Chang-Deok Kim, Yusong-gu (KR); Jung-Hun Kim, Yusong-gu (KR); Jong-Il Kim, Yusong-gu (KR); Ho-Song Cho, Seo-gu (KR); Heon-Sik Lee, Yusong-gu (KR)

Correspondence Address: Richard L Byrne, 700 Koppers Building, 436 Seventh Avenue, Pittsburgh, PA, 15219-1818, US

(21) Appl. No.: 10/432,451

(22) PCT Filed: Nov. 16, 2001
(86) PCT No.: PCT/KR01/01959

(30) Foreign Application Priority Data
Nov. 22, 2000 (KR) 2000-69393

(51) Int. Cl. A61K 38/13; A61K 7/06; A61K 7/11

(52) U.S. Cl. 424/70.14; 514/9; 514/11

(57) ABSTRACT

The present invention discloses a composition comprising a cyclosporin derivative having an excellent hair revitalizing activity as an active ingredient, and more particularly, a composition comprising cyclosporin 7-thioamide produced by chemical derivatization of cyclosporin as an active ingredient for promoting hair growth.

### TECHNICAL FIELD

The present invention relates to a hair growth promoter comprising a cyclosporin derivative as an active ingredient. More particularly, the present invention relates to a hair growth promoter comprising cyclosporin 7-thioamide produced by chemical derivation of cyclosporin as an active ingredient.

### BACKGROUND ART

On average, the human scalp contains about 100,000 to 150,000 hairs. Each hair has three main stages of growth: anagen, catagen and telogen, after which the hair falls out. This hair growth cycle is repetitive and the duration of one cycle is different from other cycles, ranging approximately 3 to 6 years. Thus, the average adult normally loses about 50 to 100 hairs every day. In general, alopecia refers to a phenomenon wherein duration of the anagen growth phase is shortened and the percentage of hairs in the catagen and telogen phases increases, whereby the number of lost hairs is increased excessively and abnormally.

There are many theories to explain for loss of hair, including for example, poor blood circulation, excessive functioning of male sex hormone, excessive production and secretion of sebum, deterioration of scalp by peroxides, bacteria, etc., hereditary factors, aging, stress, etc. However, explicit mechanisms have not been revealed. Recently, the population suffering from hair loss is tending to increase, since changing dietary habits and stress imposed on individuals due to modern social environments, etc. has increased. Also, the age of the individuals affected by alopecia is dropping and furthermore, the population of female alopecia sufferers is rising.

One of preparations which are most commonly used for treatment and prevention of alopecia is one that contains minoxidil. There are two hair-regrowth agents which have received approval from the U.S. Food and Drug Administration, and minoxidil is one of those approved hair-regrowth agents. Minoxidil was originally developed as a hypertension drug for the purpose of reducing blood pressure. However, when using this drug, as a side effect, a trichogenous effect was observed and thereafter, this drug became famous as a hair-regrowth agent. Although mechanisms by which minoxidil works as a hair-regrowth agent is not clearly understood, it is inferred that minoxidil increases blood flow by expansion of blood vessels, whereby roots of hairs are supplied with more nutrition and eventually, growth of hairs are promoted.

Such a model of blood flow increase has been indirectly supported by a recent report that minoxidil enhances the expression of vascular endothelial growth factor (VEGF), a growth factor associated with vasodilatation in the dermal papilla which is a main cell making up the hair roots (Br. J. of Dermatol., 1998, 138:407-411). Also, other than the vasodilative effect of the minoxidil in the hair-restoring mechanism, it has been reported that minoxidil promotes activation of dermal papilla cells in the roots of hair incubated in vitro, and growth of hair follicles in a tissue culture of follicles in vitro (Skin Pharmacol., 1996, 9:3-8 and J. Invest. Dermatol., 1989, 92:315-320). These facts indicate that minoxidil may work directly on the roots of hair as a growth factor.

In addition, finasteride, a main component of Propecia which has started to be sold by Merck, is used for treatment of alopecia. It inhibits conversion of the male hormone testosterone into dihydrotestosterone, which is a more potent male hormone than testosterone.

On December of 1997, the 1 mg finasteride tablet was approved by the US FDA as a hair-regrowth agent for treatment of male pattern hair loss in men only, and is now commercially available. In clinical studies, it has been demonstrated to have a significant trichogenous effect. However, there has been a report that finasteride may inhibit male sexual function as a side effect (J. Am. Acad Dermatol., 1998, 39:578-589). Since neither finasteride nor minoxidil show superior effect in clinical tests, and there is concern about side effects, many researches are conducted to develop a new and improved hair-regrowth agents.

The cyclosporin family of drugs has immunosuppressive activity. It is also effective to inhibit growth of virus, fungus, protozoan, etc. and has various physiological effects such as neoprotoxicity, hepatotoxicity, hypertension, enlargement of periodontium, trichogenous effect, and so on, as side effects (Advances in Pharmacol., 1996, 35:114-246 and Drug Safety, 1994, 10:310-317). Cyclosporin A, a representative cyclosporin, is a cyclic peptide having the following Chemical Formula, which comprises 11 amino acids, including several N-methyl amino acids and D-alanine at No. 8 residue.

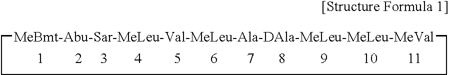

where MeBmt is N-methyl-(4R)-4-[(E)-2-butenyl]-4-methyl-L-threonine,Abu is L-α-aminobutyric acid,Sar is sarco-sine,MeLeu is N-methyl-L-leucine,Val is L-valine, Ala is L-alanine,DAla is D-alanine,MeVal is N-methyl-L-valine.

The amino acid form of cyclosporin A of the above Structure Formula 1 is L-configuration, unless otherwise specified. The residue numbering of amino acids starts from MeBmt and proceeds clockwise, i.e. 1 for MeBmt and 11 for the last MeVal (N-methyl-L-valine) as shown in the Chemical Formula 1. Nomenclature of various derivatives including cyclosporin A to Z, follows methods commonly used (Helv. Chim. Acta, 1987, 70:13-36). For example, cyclosporin B and C, in which only L-αaminobutyric acid, No. 2 residue of cyclosporin A, is substituted with L-alanine and L-threonine, respectively, are expressed by describing the different residues and the positions thereof, that is [Ala]² cyclosporin and [Thr]² cyclosporin.

Thioamide derivatives of cyclosporin, in which the carbonyl oxygen (O) of amino acid(s) of either No. 4 or No. 7 residue, or both is substituted with sulfur (S) are named as cyclosporin 4-thioamide ([⁴ψ⁵ CS—NH] cyclosporin), cyclosporin 7-thioamide ([⁷ψ⁸ CS—NH] cyclosporin), and cyclosporin 4,7-bis(thioamide) ([⁷ψ⁸ CS—NH; ⁴ψ⁵ CS—NH] cyclosporin), according to known methods (Helv. Chim. Acta 1991, 74:1953-1990; J. Org. Chem. 1993, 58:673-677; and J. Org. Chem. 1994, 59:7249-7258).

So far, possible development of cyclosporin as a hair-regrowth agent has been studied by many research groups. Particularly, researches involving animal hair regrowth tests (Arch, Dermatol. Res., 1996, 288:408-410), human alopecia areata (J. Am. Acad. Dermatol., 1990, 22:242-250), human male pattern alopecia (J. Am. Acad. Dermtol., 1990, 22:251-253 and Skin Pharmacol., 1994, 7:101-104), and inhibition effect of hair loss by chemotherapy in animal models (Clin. Lab. Invest., 1995, 190:192-196 and Am. J. Pathol., 1997, 150:1433-1441) have been widely conducted. In comparative experiments on mouser's back, it is shown that cyclosporin has a hair regrowth effect about 100 times superior to minoxidil Based on such findings, there have been attempts to utilize cyclosporin as a treatment for male pattern alopecia, and many applications for patents have been filed.

For example, Japanese Patent Publication Kokai Nos. Sho 60-243008, Sho 62-19512 and Sho 62-19513 disclose use of cyclosporin derivatives as a hair regrowth agent. Also, European Patent Publication No. 0414632 B1 discloses a cyclosporin derivative with modified No. 8 residue, PCT Patent Publication No. WO 93/17039 and PCT Patent Publication No. WO 00/51558 disclose isocyclosporin, and immunosuppressive cyclosporin derivatives, respectively. These cyclosporin and derivatives thereof are provided as a hair regrowth agent. Furthermore, in U.S. Pat. No. 5,807,820 and U.K. Patent No. 2,218,334 A, preparations containing cyclosporin with excellent transdermal absorption are suggested for new application of a hair regrowth agent.

However, there are still demands for a novel hair regrowth agent with superior hair restoring activity and no side effects.

### DISCLOSURE OF THE INVENTION

Therefore, the present invention has been made in view of the above problems, and it is an object of the present invention to provide a novel hair growth promoter having hair regrowth activity and selected from thioamide derivatives of cyclosporin having carbonyl oxygen (O) of either of amino acid(s) No. 4 or No. 7, or both substituted with sulfur (S). The thioamide. derivatives of cyclosporin substituted with sulfur have been used for studies of various derivations of cyclosporin molecules (Helv. Chim. Acta 1991, 74:1953-1990, J. Org. Chem. 1993, 58:673-677 and J. Org. Chem. 1994, 59:7249-7258). The present inventors has synthesized three thioamide derivatives of cyclosporin, cyclosporin 7-thioamide ([⁷ψ⁸ CS—NH] cyclosporin), in which the carbonyl oxygen (O) of amino acid No. 4 in the cyclosporin molecule is substituted with sulfur (S), cyclosporin 4-thioamide ([⁴ψ⁵ CS—NH] cyclosporin), in which the carbonyl oxygen (O) of amino acid No. 7 in the cyclosporin molecule is substituted with sulfur (S), and cyclosporin 4,7-bis(tioamide) ([⁷ψ⁸ CS—NH; ⁴ψ⁵ CS—NH] cyclosporin, in which the carbonyl oxygens (O) of amino acids Nos. 4 and 7 are substituted with sulfur (S), and examined for their hair regrowth effect. As a result, it was found that not all of those derivatives have hair regrowth effect, but only cyclosporin 7-thioamide ([⁷ψ⁸ CS—NH] cyclosporin, C₆₂H₁₁₁N₁₁O₁₁S) does have the hair regrowth effect.

Thus, the present invention is directed to, as a hair growth promoter, cyclosporin 7-thioamide ([⁷ψ⁸ CS—NH] cyclosporin, C₆₂H₁₁₁N₁₁O₁₁S) represented by the Chemical Formula 1.

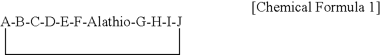

in which

A is N-methyl-(4R)-4-[(E)-2-butenyl]-4-methyl-L-threonine (MeBmt), (2S,3R,4R,6E)-3-sulfhydryl-4-methyl-2-(methylamino)-6-octenoic acid, or (2S,4R,6E)-3-oxo-4-methyl-2-(methylamino)-6-octenoic acid;

B is L-α-aminobutyric acid (Abu), L-alanine (Ala), L-threonine (Thr), L-valine (Val), or L-norvaline (Nva);

C is sarcosine, D-methylalanine ((D)—N(CH₃)—CH(CH₃)—CO—), D-2-(methylamino)pent-4-enoyl ((D)—N(CH₃)—CH(CH₂CHCH₂)—CO—), (D)-2-(methylamino)pent-4-ynoyl ((D)—N(CH₃)—CH(CH₂CCH)—CO—), or D-methylthiosarcosine (D-Sar(2-Sme), (D)—N(CH₃)—CH(SCH₃)—CO—);

D is N-methyl-L-leucine, γ-hydroxy-N-methyl-L-leucine, or L-valine;

E is L-valine, or L-norvaline;

F is N-methyl-L-leucine, γ-hydroxy-N-methyl-L-leucine, or L-leucine;

Alathio is L-alanine thioamide ([⁷ψ⁸ CS—NH], NH—CHCH₃—CS—);

G is D-alanine, or D-serine;

H is N-methyl-L-leucine, γ-hydroxy-N-methyl-L-leucine, or L-leucine;

I is N-methyl-L-leucine, γ-hydroxy-N-methyl-L-leucine, or L-leucine; and

J is N-methyl-L-valine or L-valine.

The preferred derivatives of cyclosporin of the above Chemical Formula 1 having hair regrowth activity are compounds represented by the following Chemical Formula 2.

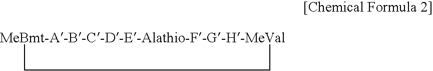

in which

MeBmt is N-methyl-(4R)-4-[(E)-2-butenyl]-4-methyl-L-threonine,

A′ is L-α-aminobutyric acid (Abu), L-alanine (Ala), L-threonine (Thr), L-valine (Val), or L-norvaline (Nva);

B′ is sarcosine, D-methylalanine ((D)—N(CH₃)—CH(CH₃)—CO—), D-2-(methylamino)pent-4-enoyl ((D)—N(CH₃)—CH(CH₂CHCH₂)—CO—), (D)-2-(methylamino)pent-4-ynoyl ((D)—N(CH₃)—CH(CH₂CCH)—CO—), or D-methylthiosarcosine (D-Sar(2-Sme), (D)—N(CH₃)—CH(SCH₃)—CO—);

C′ is N-methyl-L-leucine, γ-hydroxy-N-methyl-L-leucine, or L-valine;

D′ is L-valine, or L-norvaline;

E′ is N-methyl-L-leucine, γ-hydroxy-N-methyl-L-leucine, or L-leucine;

Alathio is L-alanine thioamide ([⁷ψ⁸ CS—NH], NH—CHCH₃—CS—);

F′ is D-alanine, or D-serine;

G′ is N-methyl-L-leucine, γ-hydroxy-N-methyl-L-leucine, or L-leucine;

H′ is N-methyl-L-leucine, γ-hydroxy-N-methyl-L-leucine, or L-leucine; and

MeVal is N-methyl-L-valine.

The more preferred thioamide derivatives of cyclosporin of the above Chemical Formula 1 having hair regrowth activity are compounds represented by the following Chemical Formula 3.

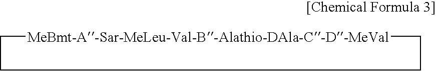

in which

MeBmt is N-methyl-(4R)-4-[(E)-2-butenyl]-4-methyl-L-threonine,

A″ is L-α-aminobutyric acid (Abu), L-alanine (Ala), L-threonine (Thr), L-valine (Val), or L-norvaline (Nva);

Sar is sarcosine;

MeLeu is N-methyl-L-leucine;

Val is L-valine;

B″ is N-methyl-L-leucine, or L-leucine;

Alathio is L-alanine thioamide ([⁷ψ⁸ CS—NH], NH—CHCH₃—CS—);

DAla is D-alanine;

C″ is N-methyl-L-leucine, or L-leucine;

D″ is N-methyl-L-leucine, or L-leucine; and

MeVal is N-methyl-L-valine.

The even more preferred 7-thioamide derivatives of cyclosporin of the above Chemical Formula 1 having hair regrowth activity are:

Cyclosporin A 7-thioamide ([⁷ψ⁸ CS—NH] cyclosporin A),

Cyclosporin B 7-thioamide ([Ala]²-[⁷ψ⁸ CS—NH] cyclosporin),

Cyclosporin C 7-thioamide ([Thr]²-[⁷ψ⁸ CS—NH] cyclosporin),

Cyclosporin D 7-thioamide ([Val]²-[⁷ψ⁸ CS—NH] cyclosporin),

Cyclosporin G 7-thioamide ([Nva]²-[⁷ψ⁸ CS—NH] cyclosporin),

Cyclosporin I 7-thioamide ([Val]²-[⁷ψ⁸ CS—NH]-[Leu]¹⁰ cyclosporin),

Cyclosporin M 7-thioamide ([Nva]²-[Nva]⁵-[⁷ψ⁸ CS—NH] cyclosporin),

Cyclosporin N 7-thioamide ([Nva]²-[⁷ψ⁸ CS—NH]-[Leu]¹⁰ cyclosporin),

Cyclosporin O 7-thioamide ([Nva]²-[⁷ψ⁸ CS—NH] cyclosporin),

Cyclosporin T 7-thioamide ([⁷ψ⁸ CS—NH]-[Leu]¹⁰ cyclosporin),

Cyclosporin U 7-thioamide ([Leu]⁶-[⁷ψ⁸ CS—NH] cyclosporin),

Cyclosporin X 7-thioamide ([Nva]²-[⁷ψ⁸ CS—NH]-[Leu]⁹ cyclosporin), and

Cyclosporin Y 7-thioamide ([Nva]²-[Leu]⁶-[⁷ψ⁸ CS—NH] cyclosporin).

In the above, Ala is L-alanine, Thr is L-threonine, Val is L-valine, Nva is L-norvaline, Leu is L-leucine, and [7ψ⁸ CS—NH] is L-alanine thioamide (NH—CHCH₃—CS—). Parenthesized names of the compounds are made according to methods commonly used as previously mentioned (Helv. Chim. Acta, 1987, 70:13-36). That is, various derivatives are expressed by describing residues different from those of the original cyclosporin molecule. For example, cyclosporins B and C, in which L-α aminobutyric acid, No. 2 residue of cyclosporin A, is substituted with L-alanine and L-threonine, respectively, are expressed by describing the different residues and the positions thereof, that is [Ala]² cyclosporin and [Thr]² cyclosporin. The cyclosporin 7-thioamide is a derivative of cyclosporin, in which the carbonyl oxygen (O) of amino acid No. 7 in the cyclosporin molecule is substituted with sulfur (S), that is, [⁷ψ⁸ CS—NH] (NH—CHCH₃—CS—)cyclosporin (C₆₂H₁₁₁N₁₁O₁₁S).

In another aspect, the present invention is directed to a hair growth promoter formulated into a liquid formulation, spray, gel, paste, emulsion, cream, conditioner, or shampoo.

### BEST MODE FOR CARRYING OUT THE INVENTION

The present invention is described in detail as follows. In order to develop a novel hair regrowing agent, the present inventors synthesized various derivatives of cyclosporin and carried out the hair regrowth evaluation tests for the synthesized derivatives. As a result, it was found that cyclosporin 7-thioamide has an superior hair regrowth (restoring) effect to any other compounds.

The following Reference Example and Examples are given by way of illustration of the best mode contemplated by the inventor(s) of carrying out the invention. However, those skilled in the art will appreciate that various modifications, additions and substitutions are possible, without departing from the scope and spirit of the invention.

### REFERENCE EXAMPLE 1

Step 1: Synthesis of Acetylcyclosporin A

2.4 g (2.0 mmol) of cyclosporin A and 0.24 g (2.0 mmol) of 4-(dimethylamino)pyridine were added to 20 ml of pyridine and 20 ml of acetic anhydride, stirred for 18 hours at room temperature, and distilled under reduced pressure. To the residue was added 100 ml of methylene chloride. The residue was then washed with water and dried over dry magnesium sulfate (MgSO₄). The crude product was purified by chromatography on a silica gel column to give 2.3 g of the title compound.

Step 2-1: Synthesis of Acetylcyclosporin A 4,7-bis(thioamide)

1.8 g (1.45 mmol) of acetylcyclosporin was dissolved in 50 ml of xylene. The resulting solution was heated to 130° C. and 0.35 g (0.87 mmol) of Lawesson reagent was added in small amounts thereto. The reaction solution was stirred for 30 minutes at 130° C., cooled to room temperature, and distilled under reduced pressure to remove the solvent. To the residue was added 100 ml of methylene chloride. The residue was then washed with water and dried over dry magnesium sulfate (MgSO₄) to form a crude product. The crude product was purified by chromatography on a silica gel column to give 0.57 g of the title compound.

Step 2-2: Synthesis of Acetylcyclosporin A 4-thioamide

1.8 g (1.45 mmol) of acetylcyclosporin was dissolved in 50 ml of xylene. The resulting solution was heated to 130° C. and 0.35 g (0.87 mmol) of Lawesson reagent was added in small amounts thereto. The reaction solution was stirred for 30 minutes at 130° C., cooled to room temperature, and distilled under reduced pressure to remove the solvent. To the residue was added 100 ml of methylene chloride. The residue was then washed with water and dried over dry magnesium sulfate (MgSO₄) to form a crude product. The crude product was purified by chromatography on a silica gel column to give 0.08 g of an acetoxy compound, i.e. the title compound.

Step 3-1: Synthesis of Cyclosporin A 4,7-bis(thioamide)

0.32 g (0.25 mmol) of acetylcyclosporin A 4,7-bis(thioamide), an acetoxy compound, was dissolved in 50 ml of methanol, and 20 ml (10 mmol) of 0.5M sodium methoxide (NaOMe) in methanol was added thereto, followed by stirring for 4 hours at room temperature. The reaction solution was neutralized using acetic acid and distilled under reduced pressure to remove the solvent. To the residue was added 100 ml of methylene chloride. The residue was then washed with water and dried over dry magnesium sulfate (MgSO₄) to form a crude product. The crude product was purified by chromatography on a silica gel column and then HPLC to give 0.27 g of the title compound.

Step 3-2: Synthesis of Cyclosporin A 4-thioamide

0.2 g of acetylcyclosporin A 4-thioamide, an acetoxy compound, was dissolved in 50 ml of methanol, and 20 ml (10 mmol) of 0.5M sodium methoxide (NaOMe) in methanol was added thereto, followed by stirring for 4 hours at room temperature. The reaction solution was neutralized using acetic acid and distilled under reduced pressure to remove the solvent. To the residue was added 100 ml of methylene chloride. The residue was then washed with water and dried over dry magnesium sulfate (MgSO₄) to form a crude product. The crude product was purified by chromatography on a silica gel column and then HPLC to give 0.16 g of the title compound.

### EXAMPLE 1

Synthesis of Cyclosporin B 7-thioamide

Step 1: Synthesis of Acetylcyclosporin B

2.4 g (2.0 mmol) of cyclosporin B and 0.24 g (2.0 mmol) of 4-(dimethylamino)pyridine were added to 20 ml of pyridine and 20 ml of acetic anhydride, stirred for 18 hours at room temperature, and distilled under reduced pressure. To the residue was added 100 ml of methylene chloride. The residue was then washed with water and dried over dry magnesium sulfate (MgSO₄). The crude product was purified by chromatography on a silica gel column to give 2.3 g of the title compound.

Step 2: Synthesis of Acetylcyclosporin B 7-thioamide

1.8 g of acetylcyclosporin B was dissolved in 50 ml of xylene. The resulting solution was heated to 130° C. and 0.35 g (0.87 mmol) of Lawesson reagent was added in small amounts thereto. The reaction solution was stirred for 30 minutes at 130° C., cooled to room temperature, and distilled under reduced pressure to remove the solvent. To the residue was added 100 ml of methylene chloride. The residue was then washed with water and dried over dry magnesium sulfate (MgSO₄) to form a crude product. The crude product was purified by chromatography on a silica gel column to give 0.19 g of an acetoxy compound, i.e. the title compound.

Step 3: Synthesis of Cyclosporin B 7-thioamide

0.2 g of acetylcyclosporin B 7-thioamide, an acetoxy compound, was dissolved in 50 ml of methanol, and 20 ml (10 mmol) of 0.5M sodium methoxide (NaOMe) in methanol was added thereto, followed by stirring for 4 hours at room temperature. The reaction solution was neutralized using acetic acid and distilled under reduced pressure to remove the solvent. To the residue was added 100 ml of methylene chloride. The residue was then washed with water and dried over dry magnesium sulfate (MgSO₄) to form a crude product. The crude product was purified by chromatography on a silica gel column and then HPLC to give 0.17 g of the title compound.

### EXAMPLE 2

Synthesis of Cyclosporin C 7-thioamide

Step 1: Synthesis of Acetylcyclosporin C

2.4 g of cyclosporin C and 0.24 g (2.0 mmol) of 4-(dimethylamino)pyridine were added to 20 ml of pyridine and 20 ml of acetic anhydride, stirred for 18 hours at room temperature, and distilled under reduced pressure. To the residue was added 100 ml of methylene chloride. The residue was then washed with water and dried over dry magnesium sulfate (MgSO₄). The crude product was purified by chromatography on a silica gel column to give 2.1 g of the title compound.

Step 2: Synthesis of Acetylcyclosporin C 7-thioamide

1.8 g of acetylcyclosporin C was dissolved in 50 ml of xylene. The resulting solution was heated to 130° C. and 0.35 g (0.87 mmol) of Lawesson reagent was added in small amounts thereto. The reaction solution was stirred for 30 minutes at 130° C., cooled to room temperature, and distilled under reduced pressure to remove the solvent. To the residue was added 100 ml of methylene chloride. The residue was then washed with water and dried over dry magnesium sulfate (MgSO₄) to form a crude product. The crude product was purified by chromatography on a silica gel column to give 0.16 g of an acetoxy compound, i.e. the title compound.

Step 3: Synthesis of Cyclosporin C 7-thioamide

0.2 g (0.16 mmol) of acetylcyclosporin C 7thioamide, an acetoxy compound, was dissolved in 50 ml of methanol, and 20 ml (10 mmol) of 0.5M sodium methoxide (NaOMe) in methanol was added thereto, followed by stirring for 4 hours at room temperature. The reaction solution was neutralized using acetic acid and distilled under reduced pressure to remove the solvent. To the residue was added 100 ml of methylene chloride. The residue was then washed with water and dried over dry magnesium sulfate (MgSO₄) to form a crude product. The crude product was purified by chromatography on a silica gel column and then HPLC to give 0.15 g of the title compound.

### EXAMPLE 3

Synthesis of Cyclosporin D 7-thioamide

Step 1: Synthesis of Acetylcyclosporin D

2.49g of cyclosporin D and 0.24 g (2.0 mmol) of 4-(dimethylamino)pyridine were added to 20 ml of pyridine and 20 ml of acetic anhydride, stirred for 18 hours at room temperature, and distilled under reduced pressure. To the residue was added 100 ml of methylene chloride. The residue was then washed with water and dried over dry magnesium sulfate (MgSO₄). The crude product was purified by chromatography on a silica gel column to give 2.1 g of the title compound.

Step 2: Synthesis of Acetylcyclosporin D 7-thioamide

1.6 g of acetylcyclosporin D was dissolved in 50 ml of xylene. The resulting solution was heated to 130° C. and 0.35 g (0.87 mmol) of Lawesson reagent was added in small amounts thereto. The reaction solution was stirred for 30 minutes at 130° C., cooled to room temperature, and distilled under reduced pressure to remove the solvent. To the residue was added 100 ml of methylene chloride. The residue was then washed with water and dried over dry magnesium sulfate (MgSO₄) to form a crude product. The crude product was purified by chromatography on a silica gel column to give 0.18 g of an acetoxy compound, i.e. the title compound.

Step 3: Synthesis of Cyclosporin D 7-thioamide

0.2 g of acetylcyclosporin D 7-thioamide, an acetoxy compound, was dissolved in 50 ml of methanol, and 20 ml (10 mmol) of 0.5M sodium methoxide (NaOMe) in methanol was added thereto, followed by stirring for 4 hours at room temperature. The reaction solution was neutralized using acetic acid and distilled under reduced pressure to remove the solvent. To the residue was added 100 ml of methylene chloride. The residue was then washed with water and dried over dry magnesium sulfate (MgSO₄) to form a crude product. The crude product was purified by chromatography on a silica gel column and then HPLC to give 0.16 g of the title compound.

### EXAMPLE 4

Synthesis of Cyclosporin G 7-thioamide

Step 1: Synthesis of Acetylcyclosporin G

2.4 g of cyclosporin G and 0.24 g (2.0 mmol) of 4-(dimethylamino)pyridine were added to 20 ml of pyridine and 20 ml of acetic anhydride, stirred for 18 hours at room temperature, and distilled under reduced pressure. To the residue was added 100 ml of methylene chloride. The residue was then washed with water and dried over dry magnesium sulfate (MgSO₄). The crude product was purified by chromatography on a silica gel column to give 2.1 g of the title compound.

Step 2: Synthesis of Acetylcyclosporin G 7-thioamide

1.8 g of acetylcyclosporin G was dissolved in 50 ml of xylene. The resulting solution was heated to 130° C. and 0.35 g (0.87 mmol) of Lawesson reagent was added in small amounts thereto. The reaction solution was stirred for 30 minutes at 130° C., cooled to room temperature, and distilled under reduced pressure to remove the solvent. To the residue was added 100 ml of methylene chloride. The residue was then washed with water and dried over dry magnesium sulfate (MgSO₄) to form a crude product. The crude product was purified by chromatography on a silica gel column to give 0.15 g of an acetoxy compound, i.e. the title compound.

Step 3: Synthesis of Cyclosporin G 7-thioamide

0.2 g of acetylcyclosporin G 7-thioamide, an acetoxy compound, was dissolved in 50 ml of methanol, and 20 ml (10 mmol) of 0.5M sodium methoxide (NaOMe) in methanol was added thereto, followed by stirring for 4 hours at room temperature. The reaction solution was neutralized using acetic acid and distilled under reduced pressure to remove the solvent. To the residue was added 100 ml of methylene chloride. The residue was then washed with water and dried over dry magnesium sulfate (MgSO₄) to form a crude product. The crude product was purified by chromatography on a silica gel column and then HPLC to give 0.15 g of the title compound.

### EXAMPLE 5

Cyclosporin I 7-thioamide was synthesized following the procedures described in Example 1.

### EXAMPLE 6

Cyclosporin M 7-thioamide was synthesized following the procedures described in Example 1.

### EXAMPLE 7

Cyclosporin N 7-thioamide was synthesized following the procedures described in Example 1.

### EXAMPLE 8

Cyclosporin O 7-thioamide was synthesized following the procedures described in Example 1.

### EXAMPLE 9

Cyclosporin T 7-thioamide was synthesized following the procedures described in Example 1.

### EXAMPLE 10

Cyclosporin U 7-thioamide was synthesized following the procedures described in Example 1.

### EXAMPLE 11

Cyclosporin X 7-thioamide was synthesized following-the procedures described in Example 1.

### EXAMPLE 12

Cyclosporin Y 7-thioamide was synthesized following the procedures described in Example 1.

Formulations

Formulation 1:

Preparation of a Hair Revitalizing Tonic Containing Cyclosporin C 7-thioamide

3 hair revitalizing tonics with different contents of cyclosporin C 7-thioamide as described in Table 1 below were prepared. Respective ingredients were mixed and agitated so that solid ingredients were dissolved to form the hair revitalizing tonics in the form of a homogenous solution. The resulting tonics were examined for the hair regrowth effect in animal models according to Test Example 1 described later. In the animal test, it was shown that Composition 1, which contains 0.1% cyclosporin C 7-thioamide, has hair regrowth effect comparable to a hair revitalizing tonic containing 0.1% cyclosporin A.

TABLE 1

| Ingredients | Composition 1 | Composition 2 | Composition 3 |
|---|---|---|---|
| Ethanol | 40.0 | 40.0 | 40.0 |
| Cyclosporin C | 0.1 | 1.0 | 8.0 |
| 7-thioamide | | | |
| Tocopherol | 0.1 | 0.1 | 0.1 |
| acetate | | | |
| Salicylic | 0.3 | 0.3 | 0.3 |
| acid | | | |
| L-menthol | 0.3 | 0.3 | 0.3 |
| Tween 20 | 0.5 | 0.5 | 0.5 |
| Fragrance | Prop. amount | Prop. amount | Prop. Amount |
| Color | Prop. amount | Prop. Amount | Prop. Amount |

| | |
|---|---|
| Water | q.s. to 100 wt % |

Formulation 2:

Preparation of a Hair Revitalizing Tonic Containing Cyclosporin G 7-thioamide

3 hair revitalizing tonics with different contents of cyclosporin G 7-thioamide as described in Table 2 below were prepared. Respectives ingredients were mixed and agitated so that solid ingredients were dissolved to form the hair revitalizing tonics in the form of a homogenous solution. The resulting tonics were examined for the hair regrowth effect in animal models according to Test Example 1 described later. In the animal test, it was shown that Composition 1, which contains 0.1% cyclosporin G 7-thioamide, has hair regrowth effect comparable to a hair revitalizing tonic containing 0.1% cyclosporin A.

TABLE 2

| Ingredients | Composition 1 | Composition 2 | Composition 3 |
|---|---|---|---|
| Ethanol | 40.0 | 40.0 | 40.0 |
| Cyclosporin G | 0.1 | 1.0 | 8.0 |
| 7-thioamide | | | |
| Tocopherol | 0.1 | 0.1 | 0.1 |
| acetate | | | |
| Salicylic | 0.3 | 0.3 | 0.3 |
| acid | | | |
| L-menthol | 0.3 | 0.3 | 0.3 |
| Tween 20 | 0.5 | 0.5 | 0.5 |
| Fragrance | Prop. Amount | Prop. amount | Prop. Amount |
| Color | Prop. Amount | Prop. Amount | Prop. Amount |

| | |
|---|---|
| Water | q.s. to 100 wt % |

Formulation 3:

Preparation of a Hair Cream Containing Cyclosporin C 7-thioamide

3 hair creams with different contents of cyclosporin C 7-thioamide as described in Table 3 below were prepared. Oil phase ingredients were mixed and heated to 80° C. so that the ingredients formed a homogenous mixture. Separately, aqueous phase ingredients were mixed and heated to 80° C. so that the ingredients formed a homogenous mixture. The prepared two mixtures of different phases at 80 ° C. were combined and emulsified. The resulting emulsion was then cooled to room temperature and fragrance and colorant were added thereto to form a hair cream. At this stage, water was added to make up the volume of the hair cream.

The resulting hair creams were examined for their hair regrowth effect in animal models according to Test Example 1 described later. In the animal test, it was shown that Composition 1 described in Table 3, which contains 0.1% cyclosporin C 7-thioamide, has hair regrowth effect comparable to a hair cream containing 0.1% cyclosporin A.

TABLE 3

| Ingredients | Composition 1 | Composition 2 | Composition 3 |
|---|---|---|---|
| Paraffin | 5.0 | 5.0 | 5.0 |
| Setostearylalcohol | 5.5 | 5.5 | 5.5 |
| Petrolatum | 5.5 | 5.5 | 5.5 |
| Glycerine - | 3.0 | 3.0 | 3.0 |
| monostearate | | | |
| Polyoxyethylene | 3.0 | 3.0 | 3.0 |
| octyldodecylether | | | |
| Propylparaben | 0.3 | 0.3 | 0.3 |
| Cyclosporin C | 0.1 | 1.0 | 8.0 |
| 7-thioamide | | | |
| Glycerin | 7.0 | 7.0 | 7.0 |
| Dipropyleneglycol | 20.0 | 20.0 | 20.0 |
| Polyethyleneglycol | 5.0 | 5.0 | 5.0 |
| Water | q.s. to 100 wt % without fragrance | | |
| | and colorant | | |
| Fragrance | Prop. | Prop. | Prop. |
| | Amount | Amount | Amount |
| Colorant | Prop. | Prop. | Prop. |
| | Amount | Amount | Amount |

Formulation 4:

Preparation of a Hair Cream Containing Cyclosporin G 7-thioamide

3 hair creams with different contents of cyclosporin G 7-thioamide as described in Table 4 below were prepared. Oil phase ingredients were mixed and heated to 80° C. so that the ingredients formed a homogenous mixture. Separately, aqueous phase ingredients were mixed and heated to 80° C. so that the ingredients formed a homogenous mixture. The prepared two mixtures of different phases at 80° C. were combined and emulsified. The resulting emulsion was then cooled to room temperature and fragrance and colorant were added thereto to form a hair cream. Then, water was added to make up the volume of the hair cream.

The resulting hair creams were examined for their hair regrowth effect in animal models according to Test Example 1 described later. In the animal test, it was shown that Composition 1 described in Table 4, which contains 0.1% cyclosporin G 7-thioamide, has hair regrowth effect comparable to a hair cream containing 0.1% cyclosporin A.

TABLE 4

| Ingredients | Composition 1 | Composition 2 | Composition 3 |
|---|---|---|---|
| Paraffin | 5.0 | 5.0 | 5.0 |
| Setostearylalcohol | 5.5 | 5.5 | 5.5 |
| Petrolatum | 5.5 | 5.5 | 5.5 |
| Glycerine - | 3.0 | 3.0 | 3.0 |
| monostearate | | | |
| Polyoxyethylene | 3.0 | 3.0 | 3.0 |
| octyldodecylether | | | |
| Propylparaben | 0.3 | 0.3 | 0.3 |
| Cyclosporin G | 0.1 | 1.0 | 8.0 |
| 7-thioamide | | | |
| Glycerin | 7.0 | 7.0 | 7.0 |
| Dipropyleneglycol | 20.0 | 20.0 | 20.0 |
| Polyethyleneglycol | 5.0 | 5.0 | 5.0 |
| Water | q.s. to 100 wt % without fragrance | | |
| | and colorant | | |
| Fragrance | Prop. Amount | Prop. Amount | Prop. Amount |
| Colorant | Prop. Amount | Prop. Amount | Prop. Amount |

Formulation 5:

Preparation of a Shampoo Containing Cyclosporin C 7-thioamide

5 3 shampoos with different contents of cyclosporin C 7-thioamide as described in Table 5 below were prepared. Ingredients except for the fragrance, colorant and water were mixed and heated while being stirred so that the ingredients formed a homogenous mixture. The resulting mixture was then cooled to room temperature and fragrance and colorant were added thereto. Finally, water was added to make up the volume of the shampoo.

TABLE 5

| Ingredients | Composition 1 | Composition 2 | Composition 3 |
|---|---|---|---|
| Sodium POE | 40.0 | 40.0 | 40.0 |
| lauryl sulfuric | | | |
| acid | | | |
| (30 wt % aqueous | | | |
| solution) | | | |
| Palm oil | 3.0 | 3.0 | 3.0 |
| fatty acid | | | |
| Diethanolamide | | | |
| propylene | 2.0 | 2.0 | 2.0 |
| glycol | | | |
| Methyl | 0.2 | 0.2 | 0.2 |
| paraoxybenzoic | | | |
| acid | | | |
| Ethanol | 2.0 | 2.0 | 2.0 |
| Cyclosporin C | 1.0 | 3.0 | 10.0 |
| 7-thioamide | | | |
| Salicylic acid | 0.3 | 0.3 | 0.3 |
| L-menthol | 0.3 | 0.3 | 0.3 |
| Fragrance | Prop. Amount | Prop. Amount | Prop. Amount |
| Colorant | Prop. Amount | Prop. Amount | Prop. Amount |

| | |
|---|---|
| Water | q.s. to 100 wt % |

Formulation 6:

Preparation of a Shampoo Containing Cyclosporin G 7-thioamide

5 3 shampoos with different contents of cyclosporin G 7-thioamide as described in Table 6 below were prepared. Ingredients except for the fragrance, colorant and water were mixed and heated while being stirred so that the ingredients formed a homogenous mixture. The resulting mixture was then cooled to room temperature and fragrance and colorant were added thereto. Finally, water was added to make up the volume of the shampoo.

TABLE 6

| Ingredients | Composition 1 | Composition 2 | Composition 3 |
|---|---|---|---|
| Sodium POE | 40.0 | 40.0 | 40.0 |
| laurylsulfuric | | | |
| acid | | | |
| (30 wt % aqueous | | | |
| solution) | | | |
| Palm oil | 3.0 | 3.0 | 3.0 |
| fatty acid | | | |
| Diethanolamide | | | |
| Propylene | 2.0 | 2.0 | 2.0 |
| glycol | | | |
| Methyl | 0.2 | 0.2 | 0.2 |
| paraoxybenzoic | | | |
| acid | | | |
| Ethanol | 2.0 | 2.0 | 2.0 |
| Cyclosporin G | 1.0 | 3.0 | 10.0 |
| 7-thioamide | | | |
| Salicylic acid | 0.3 | 0.3 | 0.3 |
| L-menthol | 0.3 | 0.3 | 0.3 |
| Fragrance | Prop. Amount | Prop. Amount | Prop. Amount |
| Colorant | Prop. Amount | Prop. Amount | Prop. Amount |

| | |
|---|---|
| Water | q.s. to 100 wt % |

Formulation 7:

Preparation of a Hair Conditioner Containing Cyclosporin C 7-thioamide

3 hair conditioners with different contents of cyclosporin C 7-thioamide as described in Table 7 below were prepared. Oil phase ingredients were mixed and heated to 80° C. so that the ingredients formed a homogenous mixture. Separately, aqueous phase ingredients were mixed and heated to 80° C. so that the ingredients formed a homogenous mixture. The prepared two mixtures of different phases at 80° C. were combined and emulsified. The resulting emulsion was then cooled to room temperature and fragrance and colorant were added thereto to form a hair conditioner. Here, water was added to make up the volume of the hair conditioner.

TABLE 7

| Ingredients | Composition 1 | Composition 2 | Composition 3 |
|---|---|---|---|
| Cetanol | 3.0 | 3.0 | 3.0 |
| Self- | 2.0 | 2.0 | 3.0 |
| emulsifiable | | | |
| Glycerol- | | | |
| monostearate | | | |
| Squalene | 10.0 | 10.0 | 10.0 |
| Cyclosporin C | 1.0 | 5.0 | 10.0 |
| 7-thioamide | | | |
| Propylene glycol | 2.0 | 2.0 | 2.0 |
| Stearyldimethyl | 8.0 | 8.0 | 8.0 |
| Benzylammonium | | | |
| chloride | | | |
| (25 wt % aqueous | | | |
| solution) | | | |
| Methyl | 0.2 | 0.2 | 0.2 |
| paraoxybenzoic | | | |
| acid | | | |
| Salicylic acid | 0.3 | 0.3 | 0.3 |
| L-menthol | 0.3 | 0.3 | 0.3 |
| Water | q.s. to 100 wt % | | |
| Fragrance | Prop. amount | Prop. amount | Prop. amount |
| Colorant | Prop. amount | Prop. amount | Prop. amount |

Formulation 8:

Preparation of a Hair Conditioner Containing Cyclosporin G 7-thioamide

3 hair conditioners with different contents of cyclosporin G 7-thioamide as described in Table 8 below were prepared. Oil phase ingredients were mixed and heated to 80° C. so that the ingredients formed a homogenous mixture. Separately, aqueous phase ingredients were mixed and heated to 80° C. so that the ingredients formed a homogenous mixture. The prepared two mixtures of different phases at 80° C. were combined and emulsified. The resulting emulsion was then cooled to room temperature and fragrance and colorant were added thereto to form a hair conditioner. Here, water was added to makeup the volume of the hair conditioner.

TABLE 8

| Ingredients | Composition 1 | Composition 2 | Composition 3 |
|---|---|---|---|
| Cetanol | 3.0 | 3.0 | 3.0 |
| Self- | 2.0 | 2.0 | 3.0 |
| emulsifiable | | | |
| Glycerol- | | | |
| monostearate | | | |
| Squalene | 10.0 | 10.0 | 10.0 |
| Cyclosporin G | 1.0 | 5.0 | 10.0 |
| 7-thioamide | | | |
| Propylene glycol | 2.0 | 2.0 | 2.0 |
| Stearyldimethyl | 8.0 | 8.0 | 8.0 |
| Benzylammonium | | | |
| chloride | | | |
| (25 wt % aqueous | | | |
| solution) | | | |
| Methyl | 0.2 | 0.2 | 0.2 |
| paraoxybenzoic | | | |
| acid | | | |
| Salicylic acid | 0.3 | 0.3 | 0.3 |
| L-menthol | 0.3 | 0.3 | 0.3 |
| Water | q.s. to 1000 wt % | | |
| Fragrance | Prop.amount | Prop.amount | Prop.amount |
| Colorant | Prop.amount | Prop.amount | Prop.amount |

### TEST EXAMPLE

Test of Hair Regrowth Effects of 7-thioamide Derivatives of Cyclosporin

C57BL/6 mice (female), 42˜49 days old, were used in this test.

First of all, mice were removed of hair on their backs using an electric shaver, and weighed. The mice were divided into several groups with weights equally distributed. After one day of adaptation, cyclosporin A and cyclosporin derivatives were applied over the hair removed area once a day per each individual for 30 days. Here, the applied amount of cyclosporin A and derivatives thereof was 100 μl (0.1% w/v). The degree of hair growth were observed by naked eye. The ratio of the area where hairs newly grew to the hair removed area were determined.

As seen in Table 9 below, remarkable hair regrowth effects were seen when cyclosporin derivatives were applied, compared to the control group on which only a vehicle was applied. The degree of the hair regrowth effects are equal to that of cyclosporin A. Also, the differences of the effects among the derivatives were not significant.

Upon observing the back conditions of mice during the test period of 30 days, no peculiar skin irritations were observed in the control group and all treated groups.

TABLE 9

| | Area ratio (%) |
|---|---|
| Vehicle | 32 |
| Cyclosporin A | 87 |
| Cyclosporin B 7-thioamide | 81 |
| Cyclosporin C 7-thioamide | 87 |
| Cyclosporin D 7-thioamide | 79 |
| Cyclosporin G 7-thioamide | 88 |

In the hair regrowth agent according to the present invention, the administrated amount capable of promoting hair regrowth is 0.01 to 30%, preferably 0.1 to 10% based on total weight of composition.

Industrial Applicability

A hair growth promoter comprising cyclosporin 7-thioamide as an active ingredient according to the present invention has excellent hair growth promoting effect, leading the superior hair restoring effect.

What is claimed is:

1. A hair growth promoter comprising as an active ingredient cyclosporin 7-thioamide represented in the following Chemical Formula 1: 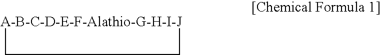
in which
A is N-methyl-(4R)-4-[(E)-2-butenyl]-4-methyl-L-threonine (MeBmt), (2S,3R,4R,6E)-3-sulfhydryl-4-methyl-2-(methylamino)-6-octenoic acid, or (2S,4R,6E)-3-oxo-4-methyl-2-(methylamino)-6-octenoic acid;
B is L-alanine (Ala), L-threonine (Thr), L-valine (Val), or L-norvaline (Nva);
C is sarcosine, D-methylalanine ((D)—N(CH₃)—CH(CH₃)—CO—), D-2-(methylamino)pent-4-enoyl ((D)—N(CH₃)—CH(CH₂CHCH₂)—CO—), (D)-2-(methylamino)pent-4-ynoyl ((D)—N(CH₃)—CH(CH₂CCH)—CO—), or D-methylthiosarcosine (D-Sar(2-Sme), (D)—N(CH₃)—CH(SCH₃)—CO—);
D is N-methyl-L-leucine, γ-hydroxy-N-methyl-L-leucine, or L-valine;
E is L-valine, or L-norvaline;
F is N-methyl-L-leucine, γ-hydroxy-N-methyl-L-leucine, or L-leucine;
Alathio is L-alanine thioamide ([⁷ψ⁸ CS—NH], NH—CHCH₃—CS—);
G is D-alanine, or D-serine;
H is N-methyl-L-leucine, γ-hydroxy-N-methyl-L-leucine, or L-leucine;
I is N-methyl-L-leucine, γ-hydroxy-N-methyl-L-leucine, or L-leucine; and
J is N-methyl-L-valine or L-valine.

2. The hair growth promoter as set forth in claim 1, wherein cyclosporin 7-thioamide as an active ingredient is represented by the following Chemical Formula 2. 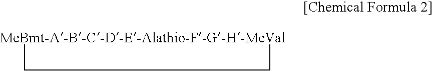
in which
MeBmt is N-methyl-(4R)-4-[(E)-2-butenyl]-4-methyl-L-threonine,
A′ is L-alanine (Ala), L-threonine (Thr), L-valine (Val), or L-norvaline (Nva);
B′ is sarcosine, D-methylalanine ((D)—N(CH₃)—CH(CH₃)—CO—), D-2-(methylamino)pent-4-enoyl ((D)—N(CH₃)—CH(CH₂CHCH₂)—CO—), (D)-2-(methylamino)pent-4-ynoyl ((D)—N(CH₃)—CH(CH₂CCH)—CO—), or D-methylthiosarcosine (D-Sar(2-Sme), (D)—N(CH₃)—CH(SCH₃)—CO—);
C′ is N-methyl-L-leucine, γ-hydroxy-N-methyl-L-leucine, or L-valine;
D′ is L-valine, or L-norvaline;
E′ is N-methyl-L-leucine, γ-hydroxy-N-methyl-L-leucine, or L-leucine;
Alathio is L-alanine thioamide ([⁷ψ⁸ CS—NH], NH—CHCH₃—CS—);
F′ is D-alanine, or D-serine;
G′ is N-methyl-L-leucine, γ-hydroxy-N-methyl-L-leucine, or L-leucine;
H′ is N-methyl-L-leucine, γ-hydroxy-N-methyl-L-leucine, or L-leucine; and
MeVal is N-methyl-L-valine.

3. The hair growth promoter as set forth in claim 1, wherein cyclosporin 7-thioamide as an active ingredient is represented by the following Chemical Formula 3. 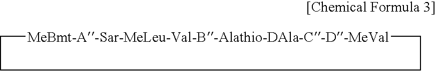
in which
MeBmt is N-methyl-(4R)-4-[(E)-2-butenyl]-4-methyl-L-threonine,
A″ is L-alanine (Ala), L-threonine (Thr), L-valine (Val), or L-norvaline (Nva);
Sar is sarcosine;
MeLeu is N-methyl-L-leucine;
Val is L-valine;
B″ is N-methyl-L-leucine, or L-leucine;
Alathio is L-alanine thioamide ([⁷ψ⁸ CS—NH], NH—CHCH₃—CS—);
DAla is D-alanine;
C″ is N-methyl-L-leucine, or L-leucine;
D″ is N-methyl-L-leucine, or L-leucine; and
MeVal is N-methyl-L-valine.

4. The hair growth promoter as set forth in claim 1, comprising cyclosporin B 7-thioamide ([Ala]²-[⁷ψ⁸ CS—NH] cyclosporin) as an active ingredient.

5. The hair growth promoter as set forth in claim 1, comprising cyclosporin C 7-thioamide ([Thr]²-[⁷ψ⁸ CS—NH] cyclosporin) as an active ingredient.

6. The hair growth promoter as set forth in claim 1, comprising cyclosporin D 7-thioamide ([Val]²-[⁷ψ⁸ CS—NH] cyclosporin) as an active ingredient.

7. The hair growth promoter as set forth in claim 1, comprising cyclosporin G 7-thioamide ([Nva]²-[⁷ψ⁸ CS—NH] cyclosporin) as an active ingredient.

8. The hair growth promoter as set forth in claim 1, comprising cyclosporin I 7-thioamide ([Val]²-[⁷ψ⁸ CS—NH]-[Leu]¹⁰ cyclosporin) as an active ingredient.

9. The hair growth promoter as set forth in claim 1, comprising cyclosporin M 7-thioamide ([Nva]²-[Nva]⁵-[⁷ψ⁸ CS—NH] cyclosporin) as an active ingredient.

10. The hair growth promoter as set forth in claim 1, comprising cyclosporin N 7-thioamide ([Nva]²-[⁷ψ⁸ CS—NH]-[Leu]¹⁰cyclosporin) as an active ingredient.

11. The hair growth promoter as set forth in claim 1, comprising cyclosporin O 7-thioamide ([Nva]²-[⁷ψ⁸ CS—NH] cyclosporin) as an active ingredient.

12. The hair growth promoter as set forth in claim 1, comprising cyclosporin T 7-thioamide ([⁷ψ⁸ CS—NH]-[Leu]¹⁰ cyclosporin) as an active ingredient.

13. The hair growth promoter as set forth in claim 1, comprising cyclosporin U 7-thioamide ([Leu]⁶-[⁷ψ⁸ CS—NH] cyclosporin) as an active ingredient.

14. The hair growth promoter as set forth in claim 1, comprising cyclosporin X 7-thioamide ([Nva]²-[⁷ψ⁸ CS—NH]-[Leu]⁹ cyclosporin) as an active ingredient.

15. The hair growth promoter as set forth in claim 1, comprising cyclosporin Y 7-thioamide ([Nva]²-[Leu]⁶-[⁷ψ⁸ CS—NH] cyclosporin) as an active ingredient.

16. The hair growth promoter in accordance with claim 1, which is formulated in a form selected from the group consisting of liquid formulation, spray, gel, paste, emulsion, cream, conditioner, and shampoo.
